(19) United States
(12) Patent Application Publication
(10) Pub. No.: US 2008/0146919 A1
(43) Pub. Date: Jun. 19, 2008

(54) Method for implanting a cardiac implant with real-time ultrasound imaging guidance

(76) Inventors: Estelle Camus, Erlangen (DE); Martin Ostermeier, Buckenhof (DE)

Correspondence Address: SCHIFF HARDIN, LLP;PATENT DEPARTMENT, 6600 SEARS TOWER, CHICAGO, IL, 60606-6473, US

(21) Appl. No.: 11/540,621

(22) Filed: Sep. 29, 2006

(51) Int. Cl. A61B 8/00

(52) U.S. Cl. 600/437

(57) ABSTRACT

In a method for implanting a cardiac implant, a 3D CT dataset of a cardiac region of interest at which an implant is to be implanted, is displayed and the implantation procedure is planned, which includes the physician electronically marking a best implantation site in the displayed image. This marking is then included in the 3D CT dataset. A 3D ultrasound dataset of the region of interest is acquired, and is brought into registration with the 3D CT dataset that incorporates the marking, and a fused image is produced therefrom. The fused image is displayed during the implantation procedure, and is updated with multiple real-time 2D ultrasound images obtained using the catheter that is employed to deliver the implant to the implantation site.

### BACKGROUND OF THE INVENTION

1. Field of the Invention

The present invention concerns a method and an apparatus for combined ultrasound and computed tomography (CT) image acquisition, particularly in the field of medicine for assisting in procedures conducted in the cath lab.

2. Description of the Prior Art

Implantable devices have recently been developed to improve perfusion of the heart. One such device is called the VPass implant and is commercially available from Percardia, Inc. This device is a stent-like device that is placed within the myocardial wall to create a tunnel between the left ventricle and a coronary vein.

Devices of this type can be implanted percutaneously in the cath lab, so that no open surgery is required. Percutaneous procedures provide many advantages, such as less trauma to the patient's body (less invasive) and a high cost reduction (shorter hospital stay, less medical personnel required), while providing the same benefit to the patient's health as other more traumatic and/or more expensive procedures. As a result; such percutaneous procedures are preferred and the number of such procedures performed yearly is increasing rapidly.

A primary difficulty with percutaneous implantation of a device within the patient's body, however, is that the implantation must proceed “blindly” because the physician does not have direct visual access to the implantation site or the implantation path to the implantation site. Therefore, guiding techniques, including guidance of puncture needles and delivery catheters, have been developed to support such percutaneous procedures.

In the example of the VPass implant, conventionally guidance for implanting that device has been accomplished using real-time IVUS (intravenous ultrasound) imaging and real-time fluoroscopic imaging.

Fluoroscopic imaging provides real-time information as to the position of the puncture/delivery catheter, which contains the IVUS catheter and the needle that has been introduced in the coronary veins. This information, however, is only in the form of a 2D projection, and thus exact guidance in three-dimensional space is not provided. Another drawback of this conventional procedure is the necessity of exposing the patient to a high radiation dose if fluoroscopic imaging is necessary over longer periods of time.

The IVUS imaging provides real-time information as to the position of the needle in two dimensions as well, and thus similarly does not provide three-dimensional information. The IVUS image shows only one cross-section of the coronary vein, and is limited as to penetration.

The three-dimensional orientation of the IVUS catheter can be derived from the 2D fluoroscopic projection, based on an extensive knowledge of the surrounding anatomy possessed by the implanting physician. Nevertheless, guidance in this conventional manner is still difficult and challenging.

### SUMMARY OF THE INVENTION

An objective of the present invention is to provide a method and apparatus for ultrasound imaging that are suitable for guiding the implantation of a cardiac implant, that at least alleviate some of the aforementioned disadvantages associated with known systems and techniques.

This object is achieved in accordance with the present invention by a method and apparatus wherein a dynamic CT cardiac dataset is acquired that shows the structures of interest (heart chamber and coronary vein) together with ECG signals. This is a 3D CT dataset. In the case of a VPass implantation, the device implantation is planned by the physician using calipers to measure the thickness of the myocardial wall in the area of the left ventricle and the distance between the left ventricle and the coronary vein using a displayed 2D image obtained from the 3D dataset. From this measurement, the physician determines the best position to implant the intracardiac device between the left ventricle and the coronary vein, using the 3D dataset. After determining the best position, the physician electronically places marks on the 3D CT datasets designating the 3D position of the intracardiac device relative to the 3D CT dataset. These marks are electronically saved and subsequently displayed together with the 3D dataset. The marks represent the best puncture point for the needle within the coronary vein and the best entry point for the needle within the myocardial wall.

2D ultrasound image datasets are acquired with an intracardiac echo (ICE) catheter, which is an ultrasound catheter, together with ECG signals. The 2D ultrasound datasets respectively represent different angulations and/or positions of the ICE catheter within the heart, and depict the structures of interest (coronary vein and left ventricle) from different viewing angles.

A 3D ultrasound dataset is reconstructed based on these 2D ultrasound acquisitions. The 3D CT dataset that was previously obtained, and the 3D ultrasound dataset, are brought into registration. The 3D CT dataset is fused with the 3D ultrasound dataset by a 3D fusion technique, and the marks that were entered in the planning phase thus exist in the fused image

Implantation of the intracardiac device then proceeds under ICE guidance, including needle puncture followed by device delivery. For this purpose, real-time 2D ultrasound datasets are acquired with the ICE catheter, and are used to update the aforementioned 3D fusion representation. This allows real-time tracking of the needle/device in the ultrasound dataset. An acoustical or optical signal can be provided to the physician when the needle position overlaps the marks that were entered in the planning phase, and/or when the position of the device delivery catheter overlaps the marks that were entered in the planning phase.

### DESCRIPTION OF THE DRAWINGS

FIG. 1 is a flowchart illustrating the basic steps and components of the inventive method and apparatus.

FIG. 2 is a flowchart illustrating the basic steps of the inventive method in more detail.

FIG. 3 is a flowchart illustrating the implantation step of FIG. 2 in more detail.

### DESCRIPTION OF THE PREFERRED EMBODIMENTS

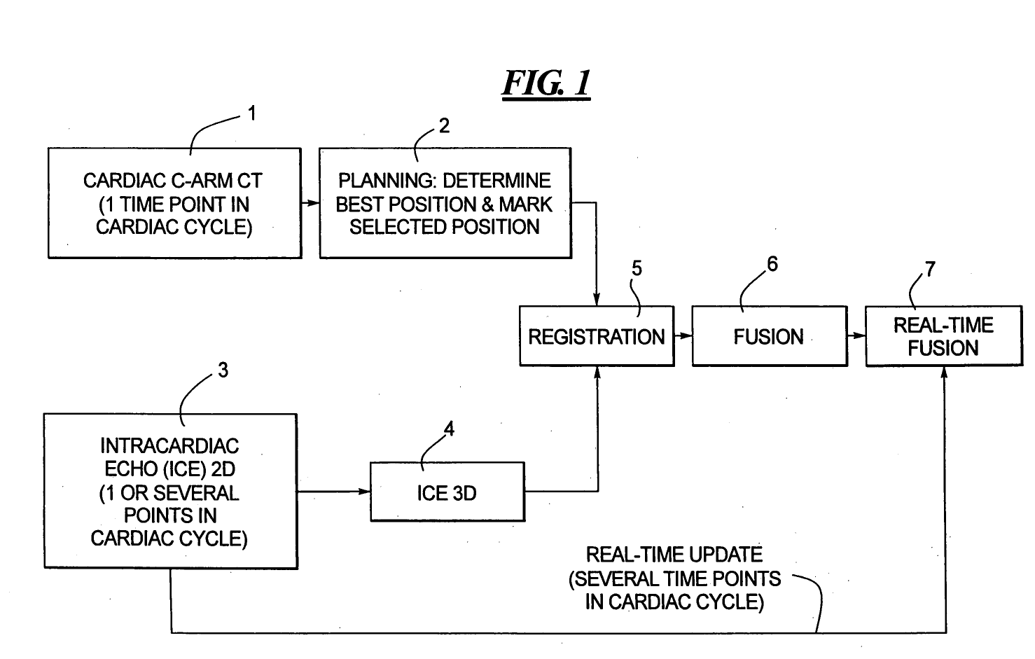

In the schematic workflow diagram shown in FIG. 1 for illustrating the inventive procedure, computed tomography (CT) data are acquired of the cardiac region of a patient at one point in time of the cardiac cycle. The CT data can be acquired using a C-arm-CT system, which is commercially available from Siemens Medical Solutions. Such a C-arm-CT system delivers CT-like images during angiography and treatment planning procedures.

The image represented by the CT data encompasses the structures of interest, such as a particular heart chamber and the coronary vein. The CT data are acquired together with ECG signals from the patient. It may be necessary to acquire two or more sets of such CT data in order to display the complete anatomy of interest. If so, all of the acquired CT datasets are then fused together to represent the structures of interest in one 3D CT dataset.

Block 2 in FIG. 1 schematically illustrates the planning phase wherein, based on the displayed 3D dataset acquired in block 1, the physician measures, such as by using calipers, the thickness of the myocardial wall in the area of the left ventricle and the distance between the left ventricle and the coronary vein, for the example of implanting a VPass implant, as described above. Based on this measured information, the physician determines the size of the device to be implanted, and selects an appropriate implant from an inventory of available implants.

Also in the planning phase encompassed within block 2, the physician determines the best position to implant the intracardiac device between the left ventricle and the coronary vein. For this purpose, 2D and 3D representations of previously acquired CT data (gained from CT or C-arm-CT) are used simultaneously. At the end of the planning phase, the physician enters marks on the datasets, by electronic interaction with the displayed image. These marks indicate a 3D position at which the implantation of the implant will occur, and can be saved together with the dataset and subsequently displayed within the dataset. These marks represent the best puncture point for the needle within the coronary vein, and the best entry point for the needle within the myocardial wall.

As also shown in FIG. 1, 2D ultrasound datasets are acquired with an intracardiac ultrasound catheter (intracardiac echo, or ICE, catheter) together with ECG signals. This can occur at one or several points in the cardiac cycle. The different datasets correspond to different positions of the ICE catheter within the heart, and depict the structures of interest (coronary vein and left ventricle) from different viewing angles.

In block 4 of FIG. 1, a 3D ultrasound dataset is reconstructed based on the 2D acquisitions made in block 3.

In block 5, the CT dataset is brought into registration with the 3D ultrasound dataset, using the ECG signals that were respectively required with each of these datasets.

In block 6 a 3D fusion of the 3D CT dataset with the 3D ultrasound dataset (that were brought into registration with each other in block 5) occurs. This fused dataset embodies the marks that were made in planning phase in block 2.

In block 7 in FIG. 1, the fused image generated in block 6 is displayed and is used in the implantation procedure. This displayed, fused image shows the marks that were made in the planning stage and is a real-time image also showing the progress of the delivery catheter through a vein to the implant site. For this purpose, the fused image is updated in real time by the acquisition of real-time 2D ultrasound images with the ICE catheter, occurring again in block 3. These real-time 2D ultrasound image datasets acquired in block 3 are fused with the displayed image in block 7 during the procedure, allowing real-time tracking of the needle/implant in the displayed image. As indicated in FIG. 1 the real-time updating can occur at several points in time in each cardiac cycle. Reconstruction of the 3D ultrasound dataset using the real-time 2D ultrasound datasets can be performed in accordance with the techniques described in EP 0 961 135 B1, or by the use of a position sensor located at the tip of the ICE catheter.

For the registration in block 5, any of several registration techniques can be used. A 2D/3D registration technique can be used, wherein some of the 2D ultrasound datasets (for example, two or three) are registered within the 3D CT dataset using landmarks, manual fit, or automatic fitting using image processing, such as by automatic segmentation of anatomical structures. The entire 3D ultrasound dataset (containing those registered 2D datasets) then is fitted within the 3D CT dataset.

Another alternative is 3D/3D registration, which achieves the best volume fit.

Another alternative is fluoro registration, wherein a fluoroscopic dataset (two or more images) showing the tip of the ICE catheter is acquired, and the position of the ICE catheter tip is determined from this dataset. Only the orientation of the ultrasound dataset along the longitudinal axis of the catheter is not known. The 3D CT dataset is inherently registered to the fluoro images, and by using this co-registration and a best-fit algorithm to find the orientation of the ultrasound data set within the 3D CT dataset, the ultrasound dataset can be completely registered with the 3D CT dataset.

Another alternative is to use a position sensor located at the tip of the delivery catheter.

If the 2D ultrasound datasets are respectively acquired at several points in time in the cardiac cycle, the simultaneously acquired ECG signal are used to select those 2D ultrasound datasets that correspond to the same ECG phase as the 3D CT dataset.

Since the CT dataset was acquired at one point in time within the cardiac cycle, this dataset remains static over time while the ultrasound datasets, acquired at several points in time within the cardiac cycle, are displayed at the times received, correlated according to the ECG signals.

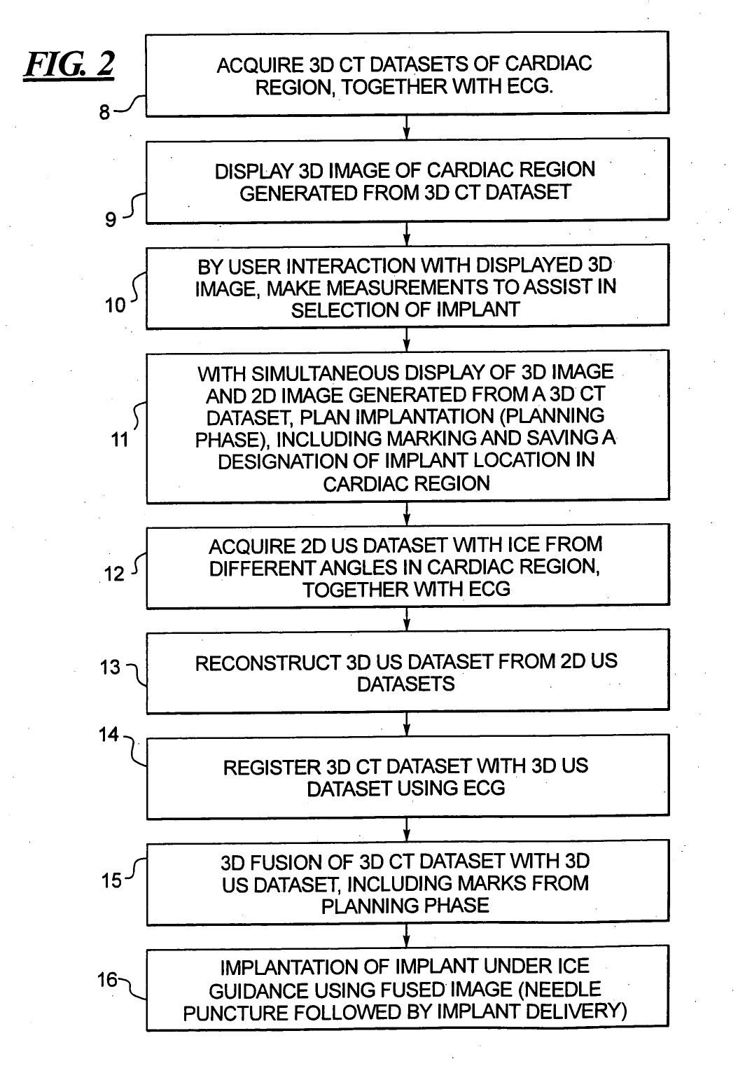

A detailed flowchart of the procedure described in the context of FIG. 1 is shown in FIG. 2.

In block 8, 3D CT datasets are acquired of the cardiac region of interest, together with an ECG.

In block 9, this 3D image of the cardiac region of interest is displayed at a computerized display that allows electronic user interaction with the displayed 3D image.

In block 10, the physician makes the aforementioned measurements on the displayed image in the planning phase.

In block 11, with simultaneous display of the 3D image and a 2D image generated from a 3D CT dataset, the implantation is planned, including marking and saving the aforementioned designations of the implant location in the cardiac region.

In block 12, multiple 2D ultrasound datasets are acquired with an ICE catheter respectively from different viewing angles of the region of interest, together with the ECG signal.

In block 14, the 3D CT dataset is brought into registration with the 3D ultrasound dataset, using the respective ECG signals that were obtained with each dataset.

In block 15, a 3D fusion of the 3D CT dataset with the 3D ultrasound dataset (that have now been brought into registration with each other) occurs. The fused image includes the marks that were entered into the 3D CT dataset during the planning phase.

In block 16, the implantation of the implant takes place under ICE guidance, using the fused image. This implantation includes the needle puncture followed by the implant delivery.

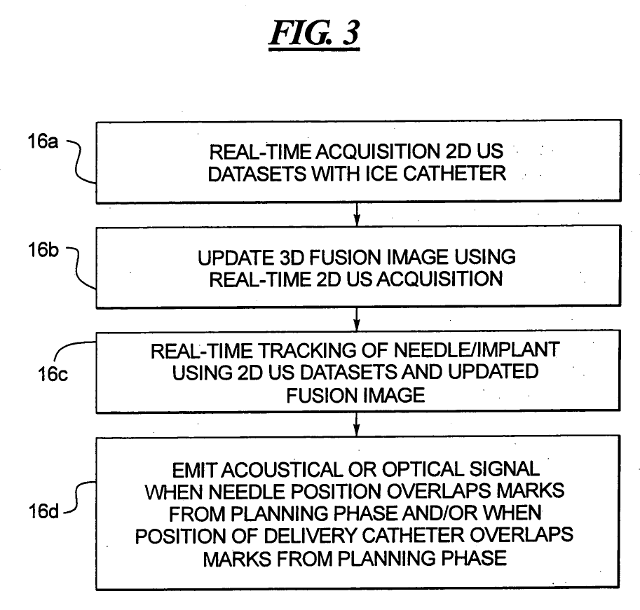

FIG. 3 shows details of the implantation procedure represented by block 16 in FIG. 2. In block 16a shown in FIG. 3, the aforementioned real-time acquisition of 2D ultrasound datasets with the ICE catheter takes place. In block 16b, the 3D fusion image is updated using these real-time 2D ultrasound acquisitions. In block 16c, real-time tracking of the needle/implant takes place, using the 2D ultrasound datasets and the updated fusion image.

As indicated in block 16d, an acoustical or optical signal can be emitted when the needle position overlaps the marks from the planning phase and/or when the position of the delivery catheter overlaps the marks from the planning phase. This provides a perceptible indication to the physician during the procedure that proper positioning of the implant has been achieved.

The method described above can be implemented in a computerized control system that operates components, such as a C-arm CT system and an ICE ultrasound system, that are conventionally present in a standard cath lab, in accordance with the above steps. The computerized control unit can be operated by a computer readable medium encoded with program code to implement the above steps, including the steps involving image processing.

The technique described herein allows time-resolved 3D ultrasound data to be used during the planning phase to predict the behavior/movement of the device to be implanted. Moreover, the radiation dose to which the patient is exposed can be reduced to a minimum by acquiring (at best) only one CT dataset and perform the rest of the procedure using the ICE, since the use of fluoroscopic imaging is only optional (as one of the possible registration alternatives). The CT data and the ultrasound data provide complementary information regarding the anatomical structures. The CT data provides very good spatial resolution, and thus very good geometrical representation of the different structures relative to each other, while the ultrasound data provides real-time information (time resolution) regarding these structures and their mechanical properties, but with less spatial resolution.

Although modifications and changes may be suggested by those skilled in the art, it is the intention of the inventors to embody within the patent warranted hereon all changes and modifications as reasonably and properly come within the scope of their contribution to the art.

1. A method for implanting a cardiac implant, comprising the steps of:
at one point in time in a cardiac cycle of a heart, acquiring a 3D CT dataset of a cardiac region of interest at which an implant is to be implanted;
at an electronic display, displaying an image of said cardiac region of interest represented by said 3D CT dataset and planning implantation of said implant by manual electronic interaction with said image, including making at least one mark in said region of interest at said display associated with an implant site of said implant, and electronically incorporating said at least one mark into said 3D CT dataset;
acquiring a 3D ultrasound dataset representing at least a portion of said region of interest;
electronically bringing said 3D CT dataset, with said at least one mark incorporated therein, and said 3D ultrasound dataset, into registration;
fusing the 3D CT dataset and the 3D ultrasound dataset, in registration with each other, to obtain a fused image that includes said at least one mark;
electronically displaying said fused image; and
percutaneously implanting said implant with a delivery system by guiding said delivery system to the implant site based on said at least one mark in the displayed fused image, while obtaining real-time 2D ultrasound images of said region of interest at multiple times in respective cardiac cycles, and updating the displayed fused image with said real-time 2D ultrasound images.

2. A method as claimed in claim 1 wherein the step of acquiring a 3D CT dataset comprises three-dimensionally acquiring said 3D CT dataset.

3. A method as claimed in claim 1 wherein the step of planning implantation of said implant comprises manually making a measurement at said image of said region of interest displayed at said display, and selecting an implant of an appropriate size, from among a plurality of available implants of respectively different sizes, based on said measurement.

4. A method as claimed in claim 3 wherein said implant is a stent to be implanted in a myocardial wall of the heart to communicate the left ventricle of the heart with the coronary vein, and wherein the step of making a measurement comprises measuring a thickness of the myocardial wall and a distance between the left ventricle and the coronary vein.

5. A method as claimed in claim 4 wherein the step of making at least one mark in said region of interest at said display comprises making a mark at said region of interest in said display indicating a best position to implant said stent between the left ventricle and the coronary vein.

6. A method as claimed in claim 5 wherein the step of making at least one mark comprises making a first mark in said region of interest indicating a puncture point for a needle within the coronary vein and a second mark indicating a best entry point for said needle entering the left ventricle.

7. A method as claimed in claim 1 wherein the step of acquiring a 3D ultrasound dataset comprises acquiring a plurality of 2D ultrasound datasets respectively from different points of view of said region of interest, and combining said plurality of 2D ultrasound datasets to form said 3D ultrasound dataset.

8. A method as claimed in claim 7 wherein the step of acquiring a plurality of 2D ultrasound datasets comprises acquiring a plurality of 2D ultrasound datasets with an intracardiac echo catheter.

9. A method as claimed in claim 7 wherein the step of bringing said 3D CT dataset into registration with said 3D ultrasound dataset comprises bringing multiple, but less than all, of said plurality of 2D ultrasound datasets into registration with said 3D CT dataset, and bringing said 3D ultrasound dataset into registration with 3D CT dataset based on said multiple 2D ultrasound datasets in registration with said 3D CT dataset.

10. A method as claimed in claim 7 comprising acquiring said plurality of 2D ultrasound images with an intracardiac echo catheter having a catheter tip, and wherein the step of bringing said 3D CT dataset into registration with said 3D ultrasound dataset comprises acquiring a fluoroscopic dataset, showing said catheter tip, with the same imaging apparatus for acquiring said 3D CT dataset, so that said fluoroscopic dataset and said 3D CT dataset are inherently in registration, automatically electronically determining a position of said catheter tip in said 3D CT dataset from said fluoroscopic dataset, and bringing said 3D CT dataset into registration with said 3D ultrasound dataset using said position of said catheter tip and a best fit algorithm.

11. A method as claimed in claim 7 comprising acquiring said plurality of 2D ultrasound images with an intracardiac echo catheter having a catheter tip with a position sensor, from different viewing angles of said region of interest, and bringing said 3D CT dataset into registration with said 3D ultrasound dataset using position information obtained from said position sensor.

12. A method as claimed in claim 1 wherein the step of bringing said 3D CT dataset into registration with said 3D ultrasound dataset comprises using a 2D/3D best volume fit technique.

13. A method as claimed in claim 1 comprising generating a humanly perceptible signal, selected from the group consisting of optical signals and audio signals, when said implant in at least one of said real-time 2D ultrasound images overlaps said at least one mark in the displayed fused image.

14. A method as claimed in claim 1 wherein said delivery system employs a delivery catheter having a catheter tip, and comprising generating a humanly perceptible signal, selected from the group consisting of optical signals and audio signals, when said catheter tip in at least one of said real-time 2D ultrasound images overlaps said at least one mark in the displayed fused image.
